(19) United States
(12) Patent Application Publication
(10) Pub. No.: US 2004/0002537 A1
(43) Pub. Date: Jan. 1, 2004

(54) Use of statins in the treatment of autoimmune disease

(76) Inventors: Scott S. Zamvil, Palo Alto, CA (US); Lawrence Steinman, Stanford, CA (US); Sawsan Youssef, Palo Alto, CA (US); Olaf Stuve, San Francisco, CA (US)

Correspondence Address: BOZICEVIC, FIELD & FRANCIS LLP, 200 MIDDLEFIELD RD, SUITE 200, MENLO PARK, CA, 94025, US

(21) Appl. No.: 10/404,922

(22) Filed: Mar. 31, 2003

Related U.S. Application Data
(60) Provisional application No. 60/368,803, filed on Mar. 29, 2002.

(51) Int. Cl. A61K 31/401; A61K 31/366; A61K 31/225

(52) U.S. Cl. 514/423; 514/460; 514/548

(57) ABSTRACT

Methods are provided for the treatment of autoimmune disease, in particular multiphasic autoimmune disease, by administering statins. Of particular interest is the administration of statins during an ongoing disease.

### INTRODUCTION

The complexity of the immune system has been a daunting barrier to an understanding of immune system dysfunction. In recent years, the techniques of molecular biology have provided insight into the mechanisms and components that underlie immunity. To a large extent, the story of immunity is the story of lymphocytes. Lymphocytes possess an extremely complex and subtle system for interacting with each other, with antigen-presenting cells, and with foreign antigens and cells.

Multiple Sclerosis (MS) is the most common central nervous system (CNS) demyelinating disease, affecting 350,000 (0.1%) individuals in North America and 1.1 million worldwide. In general, MS is considered to be an autoimmune disease mediated in part by proinflammatory CD4 T (Th1) cells that recognize specific myelin proteins in association with MHC class II molecules expressed on antigen (Ag) presenting cells (APC). Similar to other autoimmune diseases, MS susceptibility is genetically linked to the MHC HLA-D region (HLA DR2(DRβ*1501, DQβ*0602).

MS is multiphasic. Attacks of neurologic impairment occur in the early phase, which is characterized histologically by inflammatory lesions containing a predominance of CD4 T cells, B cells and both MHC class II positive macrophages and microglia, a resident CNS antigen presenting cell (APC). After multiple acute attacks a chronic “secondary progressive” phase with sustained neurologic impairment often ensues. This “irreversible” phase is characterized by neuronal loss and atrophy.

In the U.S., two IFN-β medications, avonex (IFN-β1a) and betaseron (IFN-β1b), and copaxone (glatiramer acetate) have been approved for treatment of the early inflammatory “relapsing-remitting” phase. The IFN β's exert several effects in an Ag-nonspecific manner while copaxone appears to preferentially affect T cells specific for CNS autoantigens. Novantrone, a cancer chemotherapeutic agent that interferes with DNA repair, has been approved for treatment of secondary progressive MS. In addition to their side effects and potential toxicities, these medications are only partially effective, underscoring the need to develop new immunomodulatory MS therapies.

Approved for their cholesterol lowering effects in prevention of atherogenesis, evidence suggests that the 3-hydroxy-3-methylglutaryl coenzyme A (HMG-CoA) reductase inhibitors known as “statins” may be beneficial in treatment of inflammatory diseases. In 1995, it was reported that pravastatin treatment of cardiac transplant patients was associated with a reduction in hemodynamically significant rejection episodes and increased survival, independent of its cholesterol lowering effects. Metabolites of mevalonate, the product of HMG-CoA reductase, were known to be involved in post-translational modification (isoprenylation) of specific proteins involved in signal transduction and cell differentiation. However, greater appreciation for the potential immunodulatory effects of statins developed when it was demonstrated that lovastatin inhibited production of nitric oxide synthase (iNOS) and proinflammatory cytokines (TNFα, IL-1β and IL-6) by microglia and astrocytes, another CNS APC. The observation that statins inhibited iNOS secretion suggested they might also have neuroprotective effects.

Statins prevented IFN-γ-inducible class II expression on nonprofessional APC by inhibiting transcription at the IFN-γ-inducible promoter (p) pIV of the MHC class II transactivator (CIITA), the master regulator for class II expression, but did not alter constitutive expression in dendritic cells, which utilize pI or B cells, which use pIII. Thus, statins may suppress Ag presentation by nonprofessional resident CNS APC.

Statins inhibit lymphocyte secretion of matrix metalloprotease-9 (MMP-9), an enzyme involved in basement membrane degradation and transmigration across endothelial barriers, including the blood brain barrier. Independent of HMG-CoA reductase inhibition, statins bind lymphocyte function-associated antigen-1 (LFA-1), a β2-integrin, and prevent interaction with its ligand, ICAM-1, and T cell activation. These observations suggest that statins may have beneficial effects at multiple steps in the pathogenic cascade of MS. In contrast with current MS treatments, which are administered parenterally, statins are given orally and are well tolerated. As statins appear to have different activities than currently approved MS treatments, they may also be useful in combination therapy, in addition to being considered as candidates for monotherapy.

### SUMMARY OF THE INVENTION

Methods are provided for the treatment of autoimmune disease, in particular multiphasic autoimmune disease, such as the demyelinating autoimmune diseases EAE and multiple sclerosis, by administering statins. Of particular interest is the administration of statins during an ongoing disease, for example after an initial period of disease; during remission; during a recurring disease incident; and the like. It is shown that statins are able to successfully reverse paralysis in relapsing demyelinating disease when treatment is initiated after the first attack. These drugs are shown to have pleiotropic immunomodulatory effects involving both APC and T cell compartments. Currently approved treatments for demyelinating diseases, such as multiple sclerosis, are administered parenterally, are only partly effective and are often limited by side-effects or toxicities. Statins are administered orally, are well tolerated and generally considered safe.

### BRIEF DESCRIPTION OF THE DRAWINGS

FIG. 1. EAE prevention and treatment by oral atorvastatin. Treatment at onset of MOG p35-55-induced EAE in C57BI/6 mice prevents clinical worsening (A, 7 mice in each group), while treatment after onset ameliorates EAE (B, 14 mice in each group). Treatment at onset of PLP p139-151-induced EAE in SJL/J mice prevents relapses (C, 10 mice in each group), while treatment begun after acute EAE reverses relapsing EAE (D, 10 mice in each group). prevention of acute EAE of MBP Ac1-11 induced in MBP Ac1-11 Tg mice (E, 6 mice in each group). Horizontal bars beneath each graph indicate atorvastatin treatment period. Mean EAE score are plotted against the number of days since EAE induction.

FIG. 2. Atorvastatin treatments decrease mononuclear infiltration in brains.

FIG. 3. Atorvastatin downregulates the expression of the different CIITA transcripts in vivo in the CNS. 3 groups of SJL mice were treated with: 1 mg/kg or 10 mg/kg atorvastatin or only PBS for 12 days. Two days after the beginning of the treatment EAE was induced in those mice using PLP139-151/CFA. At day 12 of the treatment (that equals day 10 of EAE) 2 mice of each group and two naive mice were sacrificed. Total RNA was extracted from the brains and total CIITA expression and specific CIITA expression was analyzed using Real Time PCR technique. (A) Shows the total expression of all CIITA mRNA transcripts (demonstrated as the internal transcripts), (B) shows the specific expression of the form I or as designed promoter I (PI, specific for dendritic cells), (C) shows the specific expression of the form III or as designed promoter III (PIII, specific for B cells) and (D) shows the specific expression of the form IV or as designed promoter IV (P IV, the IFN-γ inducible form and specific for Microglia cells). Mean transcripts copies are plotted against the treated groups. Asterisks indicate a statistically significant difference ≦0.05 by one way ANOVA test) comparing the atorvastatin treated or naive groups versus the PBS treated group in each case.

FIG. 4. Atorvastatin suppresses proliferation and promotes Th2 cytokine bias. (A) Proliferative responses of PLP p139-151-stimulated spleen cells from atorvastatin-treated and vehicle-treated PLP p139-151 immunized mice. Atorvastatin treatment is associated with diminished secretion of IL-2 (b) and IFN-γ (c), and increased production of IL-4 (D) and IL-10 (at 10 mg/kg atorvastatin) (E). Proliferation was measured by ³H-thymidine incorporation, and cytokine measurements by ELISA.

FIG. 5: (A) An anti-phospho STAT6-specific Western was done in order to determine the extent of STAT6 activation in mice treated with PBS (lane 1), 1 mg/kg atorvastatin (lane 2), 10 mg/kg atorvastatin (lane 3), or mrIL-4 (10 ng/ml) treated lymphocytes (lane 4). Samples were obtained from protein lysates of draining lymph node cells from the different groups of mice. As seen in the positive control (lane 4) IL-4 treatments and Atorvastatin treatment s activate an expected ≈105 kDa isoform of STAT6 in lymph node cells. (B and C). The same blot was stripped and reprobed with antibodies against Stat6 (B) or mouse CD3 (C) as a control to ensure equal loading of each lane. The data shown are representative of two separate Western blots performed on each of two independent experiments. Molecular weights are indicated in kilodaltons.

### DESCRIPTION OF THE SPECIFIC EMBODIMENTS

Methods are provided for the treatment of autoimmune disease; particularly Th1 mediated disease, and more particularly multiphasic autoimmune disease, by administering an effective dose of a statin. The statin may be administered before, during or after the onset of disease. While the subject methods are used for prophylactic or therapeutic purposes, of particular interest is the administration of statins after onset of the disease, for example during remission; during a recurring disease incident; and the like. It is shown that statins are able to successfully reverse paralysis resulting from relapsing demyelinating disease, when treatment is initiated after the first attack. In one embodiment of the invention, oral atorvastatin prevents or reverses chronic and relapsing paralysis.

While the invention should not be limited to the mechanism of action, it is believed that statins, e.g. atorvastatin, have a number of specific effects on the immune system and in signaling pathways. Phosphorylation of STAT6 is induced, as is secretion of Th2 cytokines (interleukin (IL)-4, IL-5 and IL-10) and TGF-β. Conversely, STAT4 phosphorylation was inhibited and secretion of Th1 cytokines (IL-2, IL-12, interferon (IFN)-γ and tumor necrosis factor (TNF-α) is suppressed. Statins promote differentiation of Th0 cells into Th2 cells. In adoptive transfer, these Th2 cells can protect recipients from disease induction.

Statins also reduce CNS infiltration and major histocompatibility complex (MHC) class II expression. Treatment of microglia inhibits IFN-γ-inducible transcription at multiple MHC class II transactivator (CIITA) promoters and suppresses class II upregulation, as well as IFN-γ-inducible expression of CD40, CD80 and CD86 co-stimulatory molecules. L-mevalonate, the product of HMG-CoA reductase, reversed the statin's effects on antigen-presenting cells (APC) and T cells. Statin treatment of either APC or T cells suppressed antigen-specific T-cell activation.

As used herein, the term “treating” is used to refer to both prevention of disease, and treatment of pre-existing conditions. The treatment of ongoing disease, where the treatment stabilizes or reduces the undesirable clinical symptoms of the patient, is of particular interest. Such treatment is desirably performed prior to complete loss of function in the affected tissues. The subject therapy will desirably be administered during the symptomatic stage of the disease, and in some cases after the symptomatic stage of the disease, where the disease has recurring symptoms (i.e. is multiphasic). The presymptomatic, or preclinical stage will be defined as that period when there is T cell involvement at the site of disease, e.g. central nervous system, etc., but the loss of function is not yet severe enough to produce the clinical symptoms indicative of overt disease. T cell involvement may be evidenced by the presence of elevated numbers of T cells at the site of disease, the presence of T cells specific for autoantigens, the release of perforins and granzymes at the site of disease, response to immunosuppressive therapy, etc.

Statins are inhibitors of HMG-CoA reductase enzyme. These agents are described in detail, for example, mevastatin and related compounds as disclosed in U.S. Pat. No. 3,983,140, lovastatin (mevinolin) and related compounds as disclosed in U.S. Pat. No. 4,231,938, pravastatin and related compounds such as disclosed in U.S. Pat. No. 4,346,227, simvastatin and related compounds as disclosed in U.S. Pat. Nos. 4,448,784 and 4,450,171; fluvastatin and related compounds as disclosed in U.S. Pat. No. 5,354,772; atorvastatin and related compounds as disclosed in U.S. Pat Nos. 4,681,893, 5,273,995 and 5,969,156; and cerivastatin and related compounds as disclosed in U.S. Pat. Nos. 5,006,530 and 5,177,080. Additional compounds are disclosed in U.S. Pat. Nos. 5,208,258, 5,130,306, 5,116,870, 5,049,696, RE 36,481, and RE 36,520. Recently the “super statin” rosuvastatin has been commercialized. The lipophilicity of certain statins make them particularly suitable for subcutaneous delivery.

An effective dose of a statin is the dose that, when administered for a suitable period of time, usually at least about one week, and may be about two weeks, or more, up to a period of about 4 weeks, will evidence a reduction in the severity of the disease. It will be understood by those of skill in the art that an initial dose may be administered for such periods of time, followed by maintenance doses, which, in some cases, will be at a reduced dosage.

The formulation and administration of statins is well known, and will generally follow conventional usage. The dosage required to treat autoimmune disease may vary from the levels used for management of cholesterol, and in some instances will be higher doses, around about 5 fold increase over conventional dosage (where conventional dosage is intended to refer to approved dosage for management of cholesterol); around about 10 fold increase over conventional dosage, and may be as much as 20 fold increase, or more.

The statins can be incorporated into a variety of formulations for therapeutic administration. More particularly, the compounds of the present invention can be formulated into pharmaceutical compositions by combination with appropriate pharmaceutically acceptable carriers or diluents, and may be formulated into preparations in solid, semi-solid, liquid or gaseous forms, such as tablets, capsules, powders, granules, ointments, solutions, suppositories, injections, inhalants, gels, microspheres, and aerosols. As such, administration of the compounds can be achieved in various ways, including oral, buccal, rectal, parenteral, intraperitoneal, intradermal, transdermal, intracheal, etc., administration. The active agent may be systemic after administration or may be localized by the use of regional administration, intramural administration, or use of an implant that acts to retain the active dose at the site of implantation.

Those of skill will readily appreciate that dose levels can vary as a function of the specific compound, the severity of the symptoms and the susceptibility of the subject to side effects. Some of the specific compounds are more potent than others. Preferred dosages for a given compound are readily determinable by those of skill in the art by a variety of means. A preferred means is to measure the physiological potency of a given compound.

Determining the effectiveness of a regimen may utilize assays directed to determination of T cell responses. The assay may determine the level of reactivity, e.g. based on the number of reactive T cells found in a sample, as compared to a negative control from a naive host, or standardized to a data curve obtained from one or more patients. In addition to detecting the qualitative and quantitative presence of auto-antigen reactive T cells, the T cells may be typed as to the expression of cytokines known to increase or suppress inflammatory responses. It may also be desirable to type the epitopic specificity of the reactive T cells.

T cells may be isolated from patient peripheral blood, lymph nodes, or preferably from the site inflammation. Reactivity assays may be performed on primary T cells, or the cells may be fused to generate hybridomas. Such reactive T cells may also be used for further analysis of disease progression, by monitoring their in situ location, T cell receptor utilization, etc. Assays for monitoring T cell responsiveness are known in the art, and include proliferation assays and cytokine release assays.

Proliferation assays measure the level of T cell proliferation in response to a specific antigen, and are widely used in the art. In an exemplary assay, patient lymph node, blood or spleen cells are obtained. A suspension of from about 10⁴ to 10⁷ cells, usually from about 10⁵ to 10⁶ cells is prepared and washed, then cultured in the presence of a control antigen, and test antigens. The test antigens may be peptides of any autologous antigens suspected of inducing an inflammatory T cell response. The cells are usually cultured for several days. Antigen-induced proliferation is assessed by the monitoring the synthesis of DNA by the cultures, e.g. incorporation of ³H-thymidine during the last 18 H of culture.

Enzyme linked immunosorbent assay (ELISA) assays are used to determine the cytokine profile of reactive T cells, and may be used to monitor for the expression of such cytokines as IL-2, IL-4, IL-5, IL-10, γ-IFN, etc. The capture antibodies may be any antibody specific for a cytokine of interest, where supernatants from the T cell proliferation assays, as described above, are conveniently used as a source of antigen. After blocking and washing, labeled detector antibodies are added, and the concentrations of protein present determined as a function of the label that is bound.

The methods of the invention are of particular interest for the treatment of demyelinating inflammatory diseases, which include multiple sclerosis, EAE, optic neuritis, acute transverse myelitis, and acute disseminated encephalitis.

The course of disease for multiple sclerosis is highly varied, unpredictable, and, in most patients, remittent. The pathologic hallmark of MS is multicentric, multiphasic CNS inflammation and demyelination. Months or years of remission may separate episodes, particularly early in the disease. About 70% of patients of relapsing-remitting (RR) type, which is characterized by acute exacerbations with full or partial remissions. The remaining patients present with chronic progressive MS, which is subdivided further into (a) primary-progressive (PP), (b) relapsing-progressive (RP), which is a pattern combining features of RR and RP and is intermediate in clinical severity, and (c) secondary-progressive (SP), which many patients with RR progress to over time.

Clinical symptoms of MS include sensory loss (paresthesias), motor (muscle cramping secondary to spasticity) and autonomic (bladder, bowel, sexual dysfunction) spinal cord symptoms; cerebellar symptoms (eg, Charcot triad of dysarthria, ataxia, tremor); fatigue and dizziness; impairment in information processing on neuropsychological testing; eye symptoms, including diplopia on lateral gaze; trigeminal neuralgia; and optic neuritis.

The autoantigen in MS most likely is one of several myelin proteins (eg, proteolipid protein [PLP], myelin oligodendrocyte glycoprotein [MOG], MBP). Microglial cells and macrophages perform jointly as antigen-presenting cells, resulting in activation of cytokines, complement, and other modulators of the inflammatory process, targeting specific oligodendroglia cells and their membrane myelin. A quantitative increase in myelin-autoreactive T cells with the capacity to secrete IFN-gamma is associated with the pathogenesis of MS and EAE, suggesting that autoimmune inducer/helper T lymphocytes in the peripheral blood of MS patients may initiate and/or regulate the demyelination process in patients with MS.

Mammalian species that may be treated with the present methods include canines and felines; equines; bovines; ovines; etc. and primates, particularly humans. Animal models, particularly small mammals, e.g. murine, lagomorpha, etc. may be used for experimental investigations. Other uses include investigations where it is desirable to investigate a specific effect in the absence of T cell mediated inflammation.

The methods of the present invention also find use in combined therapies. The FDA has approved the long-term use of beta-interferons and glatiramer acetate, which is a synthetic form of myelin basic protein (MBP) that has fewer side effects than interferon. Other therapies include the administration of autoantigen encoding nucleic acids, peptides, and other immunosuppressive regimens. The combined use of the statins and other agents can have the advantages that the required dosages for the individual drugs is lower, and the effect of the different drugs complementary.

It is to be understood that this invention is not limited to the particular methodology, protocols, formulations and reagents described, as such may, of course, vary. It is also to be understood that the terminology used herein is for the purpose of describing particular embodiments only, and is not intended to limit the scope of the present invention which will be limited only by the appended claims.

It must be noted that as used herein and in the appended claims, the singular forms “a”, “and”, and “the” include plural referents unless the context clearly dictates otherwise. Thus, for example, reference to “a complex” includes a plurality of such complexes and reference to “the formulation” includes reference to one or more formulations and equivalents thereof known to those skilled in the art, and so forth.

Unless defined otherwise, all technical and scientific terms used herein have the same meaning as commonly understood to one of ordinary skill in the art to which this invention belongs. Although any methods, devices and materials similar or equivalent to those described herein can be used in the practice or testing of the invention, the preferred methods, devices and materials are now described.

All publications mentioned herein are incorporated herein by reference for the purpose of describing and disclosing, for example, the methods and methodologies that are described in the publications which might be used in connection with the presently described invention. The publications discussed above and throughout the text are provided solely for their disclosure prior to the filing date of the present application. Nothing herein is to be construed as an admission that the inventors are not entitled to antedate such disclosure by virtue of prior invention.

The following examples are put forth so as to provide those of ordinary skill in the art with a complete disclosure and description of how to make and use the subject invention, and are not intended to limit the scope of what is regarded as the invention. Efforts have been made to ensure accuracy with respect to the numbers used (e.g. amounts, temperature, concentrations, etc.) but some experimental errors and deviations should be allowed for. Unless otherwise indicated, parts are parts by weight, molecular weight is average molecular weight, and pressure is at or near atmospheric.

### EXPERIMENTAL

It was examined whether atorvastatin (Lipitor®) could inhibit the proinflammatory response in experimental autoimmune encephalomyelitis (EAE), a Th1 mediated central nervous system(CNS) demyelinating disease that serves as a model for multiple sclerosis (MS). Daily oral administration of atorvastatin initiated at the onset of MOG p35-55-induced chronic EAE in C57BL/6 mice reversed paralysis. Atorvastatin also ameliorated the relapses in SJL/J mice when given after the acute attack in relapsing remitting EAE induced by PLP p139-151. Acute EAE was also prevented in MBPAc1-11 treated Tg mice. Histological evaluation of brains and spinal cords taken from atorvastatin-treated mice, showed significant reduction in both the number of the perivascular lesions as well as the extent of infiltration in those lesions. CNS MHC class II transactivator (CIITA) expression, including expression of individual promoter (p) I, pIII and pIV transcripts, was reduced in atorvastatin-treated mice. Atorvastatin treatment was associated with reduction of CNS-autoantigen-specific proliferative T cell responses, decrease in IFN-γ and IL-2 secretion and increase of IL-4, and IL-10 secretion by these T cells. Thus, atorvastatin treatment promoted a Th2 bias. These results demonstrate that atorvastatin is an effective immunomodulatory agent for the treatment of demyelinating disease.

Methods:

Experimental Procedures

Animals. Female SJL/J, B10.PL and C57BL/6 mice (8 to 12-week-old) were purchased from the Jackson Laboratory (Bar Harbor, Me.). MBP Ac 1-11 transgenic (tg) TCR mice were backcrossed with B10.PL mice to obtain susceptibility to EAE. All animal protocols were approved by the Division of comparative Medicine at Stanford and in accordance with the National Institutes of Health guidelines.

Peptides. Peptides were synthesized on a peptide synthesizer (model 9050; MilliGen, Burlington, Mass.) by standard 9-fluorenylmethoxycarbonyl chemistry. Peptides were purified by HPLC. Structures were confirmed by amino acid analysis and mass spectroscopy. Peptides used in these experiments were mouse MBPAc1-11 (Ac-ASQKRPSQRHG), MOG35-55 (MEVGWYRSPFSRVVHLYRNGK), PLP139-151 (HCLGKWLGHPDKF); and HSVP16 (DMTPADALDDRDLEM)—a viral peptide used as a negative control in the proliferation and cytokine assays.

Drug Treatments. Atorvastatin (Lipitor®) tablets were obtained commercially and dissolved in PBS. Mice were subjected to oral administration of 0.5 ml Atorvastatin solution (1 or 10 mg/kg) or only PBS once daily using 18 mm feeding needles. The periods of the atorvastatin treatment are indicated in the result section.

EAE Induction. Relapsing remitting EAE was induced in SJL/J mice with 100 μg of PLP139-151 peptide, chronic progressive EAE was induced either in C57BL/6 or MBP Ac-1-11 TCR Tg mice with 100 μg of MOG35-55 peptide or 100 μg of MBP Ac1-11 peptide, respectively. All peptides were dissolved in PBS at a concentration of 2 mg/ml and emulsified with an equal volume of CFA, which consists of incomplete Freund's adjuvant supplemented with 4 mg/ml heat-killed mycobacterium tuberculosis H37Ra (Difco Laboratories, Detroit, Mich.). Mice were injected subcutaneously with 0.1 ml of the peptide emulsion. On the day of peptide immunization and 48 hr later, only C57BL/6 mice and MBP Ac-1-11 TCR Tg mice were also injected intravenously with 0.1 ml of 1 μg/ml Bordetella pertussis toxin in PBS. Mice were clinically scored as follows: 0, no paralysis; 1, tail weakness or paralysis; 2, hindlimb weakness or paralysis; 3, hindlimb paralysis and forelimb weakness; 4, hindlimb and forelimb paralysis; and 5, moribund or death.

Ag Specific Ex-vivo T Cell Proliferation Assay. Atorvastatin 1 mg/kg or 10 mg/kg or PBS daily treatments started 2 days before EAE induction in all the different strains. 10 days after EAE induction (ie 12 days after Atorvastatin treatments) draining lymph nodes and spleens were removed from control, 1 mg/kg or 10 mg/kg Atorvastatin treated SJL/J, C57BL/6 and MBP Ac1-11 transgenic mice. Lymph node cells (LNCs) or splenocytes were cultured in vitro for specific proliferative response to the specific encephalogenic peptide (PLP 139-151, MOG 35-55 or MBP Ac1-11, respectively). LNCs were prepared in 96-well microtiter plates in a volume of 0.2 ml/well at a concentration of 5×10⁶ cells/ml. The culture medium consisted of enriched RPMI (RPMI 1640 supplemented with L-glutamine [2 mM], sodium pyruvate [1 mM], nonessential amino acids [0.1 mM], penicillin [100 U/ml], streptomycin [0.1 mg/ml], 2-ME [5×10⁻⁵ M]) supplemented with 1% autologous fresh normal mouse serum with the addition of different peptides concentrations. Cultures were incubated in 37° C. in humidified air containing 5% CO₂. Cultures taken from SJL/J or C57BL/6 mice were incubated for 72h whereas cultures from MBPAc-1-11 Tg mice were incubated for 48 hours and then were pulsed for 18 hr with 1 μCi/well of [³H] thymidine. The cells were then harvested and counted in a β counter.

Cytokine Profile Determination. Lymph node cells and spleen cells from EAE donors were stimulated in vitro (2.5×10⁶ cells/ml) in 24-well plates with or without the encephalogenic peptide or with CoA as positive control. Cell culture supernatants were collected at different time points for measurements of cytokine levels: 48 hours for IL-2 , 72 hours for IFN-γ and TNF α, and 120 hours for IL-4 IL-10 and. Cytokine levels were determined using specific ELISA kits for the corresponding cytokines according to the manufacturer's protocols (PharMingen, San Diego, Calif., USA).

Total RNA Isolation. Mice were sacrificed and perfused with 20 ml of cold sterile PBS. Brains were immediately isolated and total RNA was isolated using Trizol reagent (Invitrogen) as recommended in the manufacturer protocol. The amounts of the total RNA were then measured at 260 nm.

Evaluation of CIITA promoter-specific mRNA expression by real-time (kinetic) RT-PCR.

One step RT-PCR is performed as described in Baranzini et al. (2000) J Immunol 165:6576. A master mix is prepared with 400 μM dUTP and 200 μM each of dATP, dCTP, and dGTP; 0.2 μM each oligonucleotide primer, 0.2×SYBR green in DMSO (1% final concentration); 2.5% glycerol; 1U uracyl N-glycosilase; 4 mM Mn (OAc)₂ and 5U rTth polymerase. RT-PCR parameters: initial incubation 10 min at 45° C. with activating uracyl N-glycosilase followed by RT 30 min at 60° C.; 50 cycles at 95° C. for 15s and 57° C. for 30s. β-actin is amplified from all samples as a housekeeping gene to normalize expression. A control without template is included for each primer set. For quantification, a 10-fold dilution series of a CIITA run-off transcript (10⁷ to 10² initial CIITA copies) is included in each reaction plate. Data are analyzed by software Sequence Detection Systems program and transferred to an MS Excel spread sheet for analysis. A calibration curve is generated by plotting CIITA (run-off transcript) for each 10-fold dilution against the number of cycles required for each product to exceed a preset threshold (Ct). Ct values are compared to those obtained on a standard curve. Primers for common CIITA (nt 2374-2458): 5′-GCCCACGAGACACAGCAA and 5′-TGAGCCGGGTGCCCAGGAA. 5′ (forward) promoter-specific primers: pI CIITA (pI nt 259) 5′-CCTGACCCTGCTGGAGAA; pIII CIITA(pIII nt 112): 5′-GCATCACTCTGCTCTCTAA; pIV CIITA: (pIV nt43): 5′-TGCAGGCAGCACTCAGAA. CIITA (nt 265) reverse primer for promoter-specific transcripts: 5′-GGGGTCGGCACTGTTAA. β-actin: (301-538): 5′-CGACCTGGGGATCTTCTA and 5′-TCGTGCCCTCAGCTTCCAA.

Western Blot Analysis for STAT-6 and STAT-4 Phosphorylation. Western Blot analysis was performed as described in Garren et al. (2001) Immunity 15:15, with minor modifications. Lymph nodes from control and atorvastatin-treated mice were homogenized in T-PER protein extraction buffer (Pierce,), with 20 μg/ml aprotinin, 20 μg/ml leupeptin, 1.6 mM Pefablock SC (Roche), 10 mM NaF, 1 mM Na₃VO₄ and 1 mM Na₄P₂O₇ (Sigma, St. Louis, Mo.). All procedures were handled on ice. As a positive control, lymph node cells from naive mice were isolated and cultured for one hour with mouse recombinant IL-4 (10 ng/ml) or INF-γ (100 units/ml), for STAT6 and STAT4 expression respectively. Protein concentrations were determined by BCA protein assay (Pierce). Lysate was added to 3×SDS loading buffer (Cell Signaling Technology) with 40 mM DTT. Products were separated by electrophoresis on a 4-15% SDS-PAGE gradient gel (BioRad). Pre-stained markers (Invitrogen) were used to determine MW. Gels were blotted to PVDF membranes at 100 V in 25 mM Tris, 192 mM glycine and 20% (v/v) methanol, then blocked 1 hr at RT with Tris-buffered saline (TBS) containing 0.1% Tween-20 and 5% nonfat dry milk. After washing in TBS and 0.1% Tween 20, membranes were hybridized overnight at 4° C. with anti-phospho-STAT6 Antibody or anti-phsopho-STAT4 antibody (Zymed, South San Francisco, Calif.) diluted 1:1000 in TBS, 0.1% Tween 20 and 5% BSA, the membranes were then processed by ECL Plus prebetween protocol (Amersham Life Sciences) for visualization of the bands by chemiluminescence. Membranes were stripped in 100 mM 2-mercaptoethanol, 2% (w/v) SDS and 62.5 mM Tris (pH 7.4) for 30 min at 60° C., then probed with anti-CD3ζ (Pharmingen, San Diego, Calif.) or anti Stat6 or anti Stat4 (both obtained from Santa Cruz Biotechnology, Santa Cruz, Calif.) as a control to verify equal loading amounts.

Histopathology. mice were sacrificed and perfused with 20 ml cold PBS followed by 20 ml of cold 4% paraformaldehyde. Brain and spinal cord were isolated and subjected to paraffin embedding procedure; sections were then subjected to hematoxylin and eosin-staining. Histological examination was performed on 10 sections of each mouse, and each section was evaluated on histological score without knowledge of the treatment status of the animal.

Statistical Analysis. Data are presented as mean ± SE. Significance of difference between two groups was examined using the Student t test. A value of p<0.05 was considered significant. One-way multiple range ANOVA test with significance level of p<0.05 was performed for multiple compression as well.

Results:

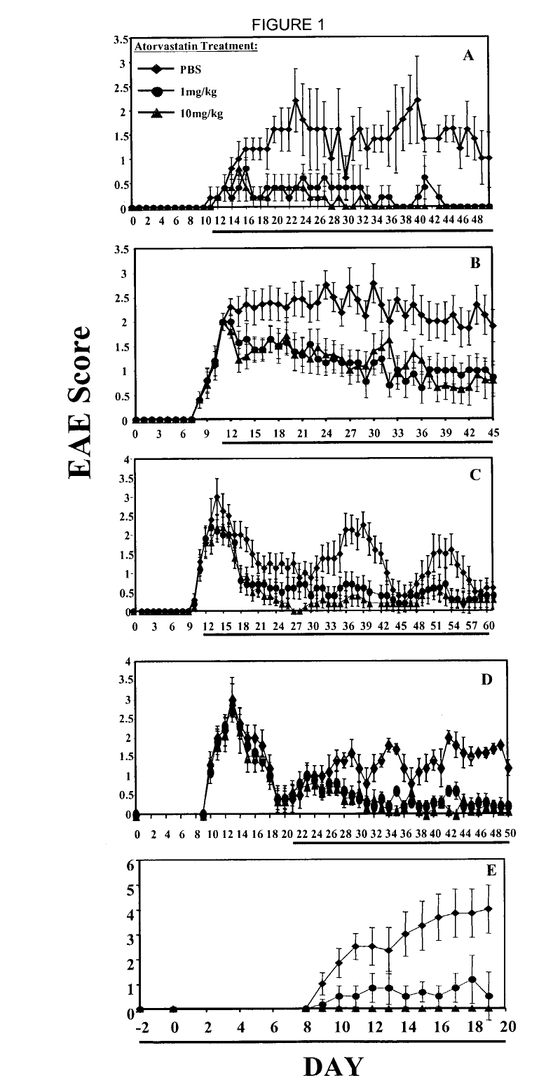

Atorvastatin reverses and prevents an on-going chronic relapsing EAE or chronic progressive EAE in mice. Initially, atorvastatin was tested for prevention of chronic EAE in C57BI/6 female mice induced by immunization with the immunodominant determinant of myelin oligodendrocyte glycoprotein (MOG), p35-55. As shown in FIG. 1A, daily oral treatment starting at the time of EAE onset with either 1 mg/kg (approximately equivalent to the highest approved adult dose of 80 mg) or 10 mg/kg atorvastatin suppressed EAE induction. Treatment after onset also ameliorated EAE (FIG. 1B). Atorvastatin treatment was tested in chronic relapsing EAE in SJL/J mice induced by immunization with encephalitogenic proteolipoprotein (PLP) peptide, p139-151. Not only was atorvastatin effective in prevention of relapsing EAE (FIG. 1C), but there was also reversal of ongoing relapsing EAE when treatment was begun after recovery from acute EAE (FIG. 1D). Atorvastatin successfully prevented acute EAE progression in MBP Ac1-11 Tg mice induced by immunization with encephalitogenic myelin basic protein(MBP) peptide, pAc1-11 (FIG. 1E).

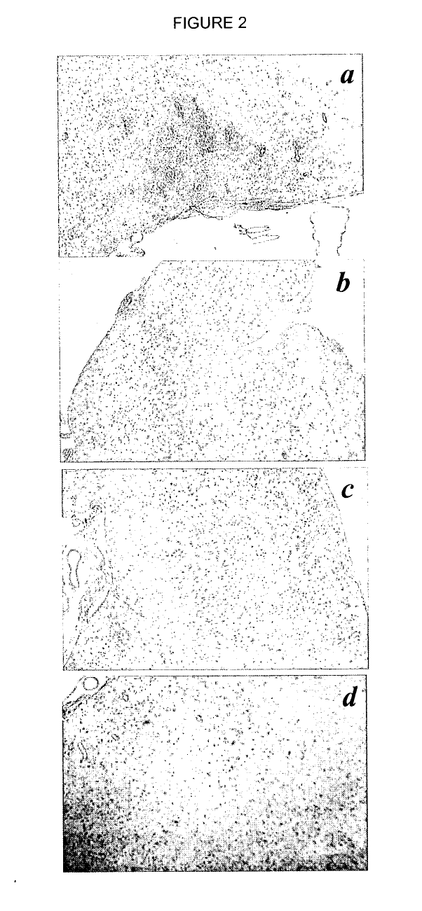

Mice from each group of all five experiments were sacrificed and brains and spinal cords were taken for CNS histological evaluation. FIG. 2 shows a representative H and E staining of brains taken from experiment A (see FIG. 1A) at day 11 after atorvastatin treatment has begun, thus 22 days after EAE induction in C57BL/6 mice. H&E sagittal brain sections taken from PBS treated C57BL/6 mice(a), from 1 mg/kg treatment (b), from 10 mg/kg treatment (c) and from naive C57BL/6 as negative control (d) sections are representative sections from 2 mice of each group.

Hematoxylin and eosin staining revealed a reduction in number and size of CNS infiltrates in atorvastatin-treated mice (shown in FIG. 2B and FIG. 2C), in comparison to PBS treated mice and naive mice (FIGS. 2A and 2D, respectively). Thus, inhibition and prevention of disease manifestation by atorvastatin oral treatments was confirmed, and demonstrated histologically at the site of inflammation (CNS). Spinal cords and brains from representative members from the other 4 animal experiments were subjected to the same analysis, and similar results obtained.

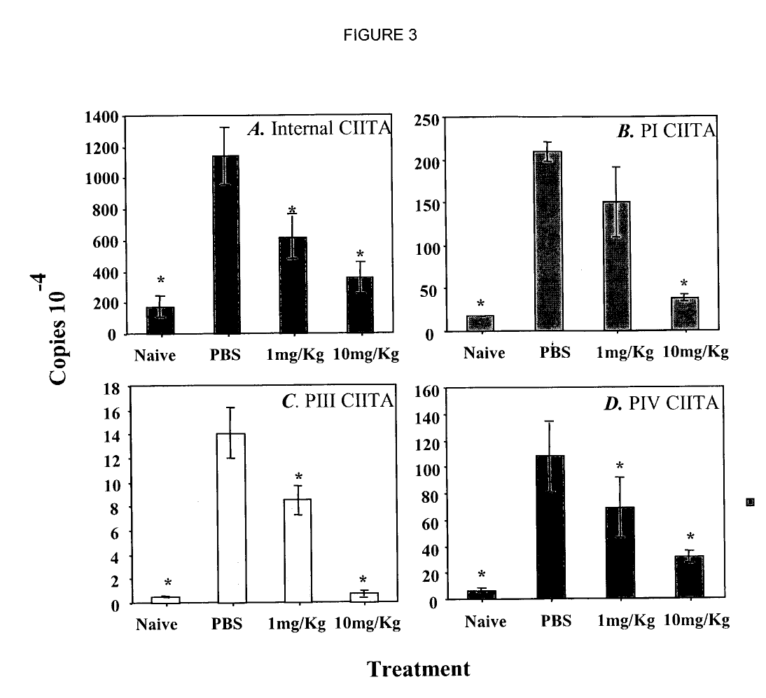

Atorvastatin downregulates CIITA expression at the site of inflammation (CNS) during EAE. In the normal central nervous system (CNS), expression of MHC class II is minimal although it is found to be highly up-regulated on microglia cells in EAE induced in mice. This expression is regulated by the factor class II transactivator (CIITA), which is required for activation of MHC class II genes especially CIITA pVI that regulate the expression in microglia cells (the major antigen presenting cells in the CNS). It was also reported that atorvastatin could inhibit the expression of MHC II, through the effect on the CIITA p IV gene. Since the histological results pointed out a reduction of infiltrates to the brains of the atorvastatin treated mice comparing with the control EAE mice, it was explored whether atorvastatin inhibits the CIITA expression in vivo in the site of inflammation. 3 groups of SJL/J mice were treated orally with either 1 mg/kg atorvastatin, 10 mg /kg or with PBS only (control). A fourth group of naive mice were added as a negative control of CIITA expression. The 3 treated groups were subjected to a daily treatments started 2 days before induction of EAE. On day 10 after the induction (12 days of the different treatments) mice from all 4 groups were sacrificed and perfused with 20 ml of cold PBS. Brains were isolated and subjected to total RNA preparation as described in the method section. RNA was subjected to real time PCR (RT-PCR) to measure the effect of atorvastatin treatments and vehicle-treated EAE on the expression of Promoter-specific CIITA transcripts at site of inflammation (CNS) in vivo. Results are demonstrated in FIG. 3.

Atorvastatin treatment inhibited the total expression of CIITA transcripts in a dose response matter (FIG. 3A). Specific CIITA analysis showed that atorvastatin treatment inhibits all three specific isoforms of CIITA (FIGS. 3B, 3C and 3D). Interestingly, atorvastatin showed a dose dependent inhibition of CIITA PIV transcript (known to be specific for regulation of MHC II expression on microglia in CNS) but also unexpectedly, PI and PIII transcripts as well, which are known to be specific for dendritic cells and B cells, respectively. The PI and PIII transcripts as shown to affected by atorvastatin in vitro.

These results demonstrate the direct inhibition of CIITA isoforms in the brain treated with atorvastatin, which could be a major factor in inhibiting the expression of MHC II in the CNS and thus preventing the massive infiltration of mononuclear cells into the CNS and reversal of EAE.

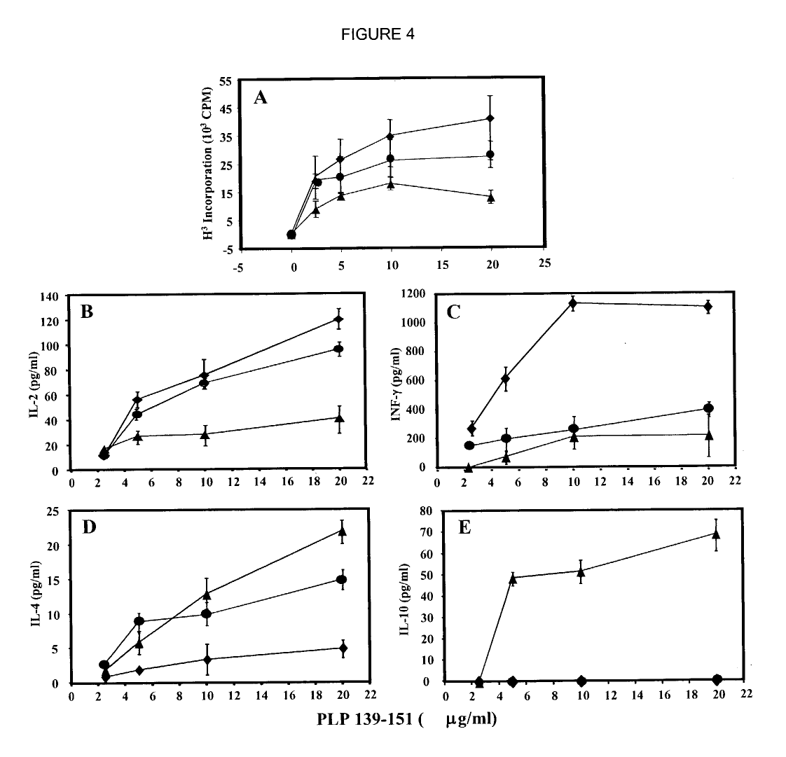

Atorvastatin promotes development of a Th2 bias. Lymphocytes isolated from spleens and lymph nodes from SJL/J female mice immunized with PLP p139-151 for EAE induction and treated with either atorvastatin or vehicle (control) were isolated after 10 days of treatment and examined for proliferation and cytokine production. As shown in FIG. 4A, PLP p139-151-specific proliferative responses were suppressed in a dose-related fashion. Production of IL-2, a Th1 cytokine, was reduced, although to a much greater extent in mice treated with 10 mg/kg (FIG. 4B). There was a dramatic reduction in secretion of IFN-γ, the hallmark Th1 cytokine, at both treatment doses (FIG. 4C). IL-4, a key anti-inflammatory cytokine, was induced at both treatment doses (FIG. 4D), while in this experiment secretion of IL-10, another anti-inflammatory Th2 cytokine, was observed at the higher treatment dose (FIG. 4E). Thus, atorvastatin suppressed Th1 cytokine production and promoted Th2 cytokines. These experiments were repeated in C57BL/6 and MBP Ac1-11-specific transgenic. Similar data were obtained. As described above for SJL/J mice, atorvastatin promoted an almost identical Th2 bias in these mice. In these in vitro experiments we have not observed increased cell death.

Atorvastatin causes activation of Stat6. In order to demonstrate that IL-4 production in the atorvastatin cause a bias from Th1 to Th2, we wanted to explore whether functional IL-4 cytokine was actually expressed during atorvastatin treatment. IL-4 is known to act through the IL-4 receptor to specifically activate STAT6, a member of the signal transducers and activators of transcription family thus it's expected to be phosphorylated when an IL-4 dependent Th2 bias occurs. SJL/J mice were daily treated with oral administrations of either atorvastatin (1 mg/kg and 10 mg/kg) or only PBS and EAE was induced, by administrating PLP/CFA, two days after the beginning of the statin treatment.

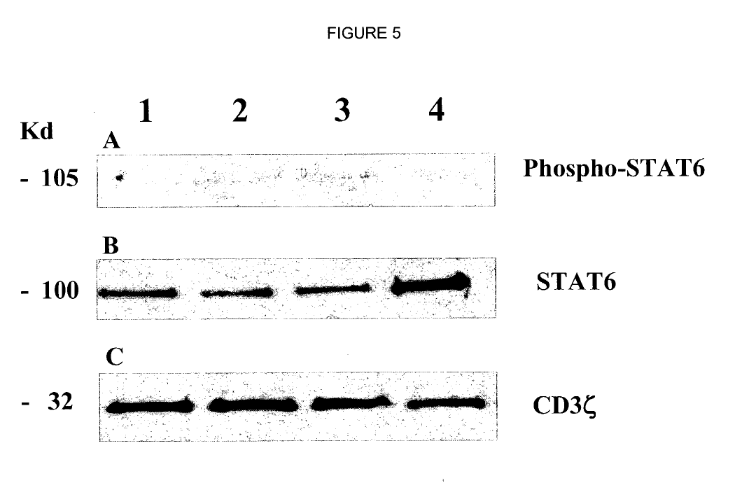

10 days after the EAE induction all groups were sacrificed and draining lymph nodes were dissected. Protein lysates were isolated from the lymph node cells and probed for the presence of activated STAT6 by Western blotting using a polyclonal antibody specific for the phosphorylated form of STAT6. As for positive control, lymph node cells were isolated from naive mice and incubated with mouse recombinant IL-4 (10 ng/ml) for one hour. Protein lysates were extracted in a similar manner. As shown in FIG. 5, phosphorylated STAT6 is seen in lymph nodes from atorvastatin treated mice (lane 2 and 3) and from the positive control (lane 4) whereas, PBS treated mice show no detectable phosphorylation of it (lane 1). The phosphorylated STAT6 identified runs at approximately 100 kDa according to pre stained markers.

What is claimed is:

1. A method of treating a multiphasic autoimmune disease, the method comprising:
administering an effective dose of a statin to a patient suffering from said multiphasic autoimmune disease;
wherein the clinical symptoms of said disease are reduced in severity.

2. The method according to claim 1, wherein said statin is administered after the initial onset of said multiphasic autoimmune disease.

3. The method according to claim 2, wherein said statin is administered during a period of remission.

4. The method according to claim 2, wherein said statin is administered during an active episode of the disease.

5. The method according to claim 1, wherein said multiphasic autoimmune disease is a demyelinating disease.

6. The method according to claim 5, wherein said demyelinating disease is multiple sclerosis.

7. The method according to claim 1, wherein said statin is selected from the group consisting of rosuvastatin, mevastatin, lovastatin, pravastatin, simvastatin, fluvastatin, atorvastatin, and cerivastatin.

8. The method according to claim 7, wherein said statin is atorvastatin.
